(19) United States
(12) Patent Application Publication
(10) Pub. No.: US 2022/0093253 A1
(43) Pub. Date: Mar. 24, 2022

(54) MENTAL HEALTH PLATFORM

(71) Applicant: Ask Rose Inc., Washington, DC (US)

(72) Inventors: Kavi Misrilall, Washington, DC (US); Atif Adam, Washington, DC (US)

(73) Assignee: Ask Rose Inc., Washington, DC (US)

(21) Appl. No.: 17/448,614

(22) Filed: Sep. 23, 2021

Related U.S. Application Data
(60) Provisional application No. 63/082,299, filed on Sep. 23, 2020.

(51) Int. Cl. G16H 50/20; G16H 10/20; G16H 40/20; G06F 40/30; G06F 40/117; G06F 40/169

(52) U.S. Cl. CPC G16H 50/20; G16H 10/20; G16H 40/20; G16H 20/70; G06F 40/117; G06F 40/169; G06F 40/30

(57) ABSTRACT

Embodiments disclosed herein generally relate to a mental health platform for clinicians and patients. A computing system generates a plurality of sets of training data. The plurality of sets of training data include portions of journal and inputs to mental health questionnaires corresponding to a plurality of patients. The computing system generates a prediction model to generate a health score of a patient, the health score indicative of the current mental health of the patient. The computing system receives input from a target patient. The input includes target responses to mental health questionnaires and target journal entries. The computing system analyzes the journal entries using natural language processing to tag portions of the journal entry with semantic tone and sentiment indicators. The computing system generates, via the prediction model, a target health score for the target patient based on the target responses and the target journal entries.

### CROSS-REFERENCE TO RELATED APPLICATIONS

This application claims priority to U.S. Provisional Application No. 63/082,299, filed Sep. 23, 2020, which is hereby incorporated by reference in its entirety.

### FIELD OF DISCLOSURE

The present disclosure generally relates to a mental health platform for clinicians and patients.

### BACKGROUND

As more research is developed, mental illness issues have become more prominent in today's society. Students, for example, are one of the most at-risk groups for mental illness and suicide. Unfortunately, identifying and destigmatizing mental illness has seen limited success.

### SUMMARY

Embodiments disclosed herein generally relate to a mental health platform for clinicians and patients. In some embodiments, a method is disclosed herein. A computing system generates a plurality of sets of training data. The plurality of sets of training data include portions of journal and inputs to mental health questionnaires corresponding to a plurality of patients. The computing system generates a prediction model to generate a health score of a patient, the health score indicative of the current mental health of the patient by injecting tags into the portions of the journal entries that signal semantic tone and sentiment of each portion of the journal entry and learning, based on modified portions of the journal entries and the inputs to the mental health questionnaires, a relationship between the journal entries, the inputs, and the mental health of the patient. The computing system receives input from a target patient. The input includes target responses to mental health questionnaires and target journal entries. The computing system analyzes the journal entries using natural language processing to tag portions of the journal entry with semantic tone and sentiment indicators. The computing system generates, via the prediction model, a target health score for the target patient based on the target responses and the target journal entries.

In some embodiments, a system is disclosed herein. The system includes a processor and a memory. The memory has programming instructions stored thereon, which, when executed by the processor, performs operations. The operations include generating a plurality of sets of training data. The plurality of sets of training data include portions of journal and inputs to mental health questionnaires corresponding to a plurality of patients. The operations further include generating a prediction model to generate a health score of a patient, the health score indicative of the current mental health of the patient, by injecting tags into the portions of the journal entries that signa semantic tone and sentiment of each portion of the journal entry and learning, based on modified portions of the journal entries and the inputs to the mental health questionnaires, a relationship between the journal entries, the inputs, and the mental health of the patient. The operations further include receiving input from a target patient. The input includes target responses to mental health questionnaires and target journal entries. The operations further include analyzing the journal entries using natural language processing to tag portions of the journal entry with semantic tone and sentiment indicators. The operations further include generating, via the prediction model, a target health score for the target patient based on the target responses and the target journal entries.

In some embodiments, non-transitory computer readable medium is disclosed herein. The non-transitory computer readable medium has instructions stored thereon, which, when executed by a processor, causes a computing system to perform operations. The operations include generating a plurality of sets of training data. The plurality of sets of training data include portions of journal and inputs to mental health questionnaires corresponding to a plurality of patients. The operations further include generating a prediction model to generate a health score of a patient, the health score indicative of the current mental health of the patient, by injecting tags into the portions of the journal entries that signal semantic tone and sentiment of each portion of the journal entry and learning, based on modified portions of the journal entries and the inputs to the mental health questionnaires, a relationship between the journal entries, the inputs, and the mental health of the patient. The operations further include receiving input from a target patient. The input includes target responses to mental health questionnaires and target journal entries. The operations further include analyzing the journal entries using natural language processing to tag portions of the journal entry with semantic tone and sentiment indicators. The operations further include generating, via the prediction model, a target health score for the target patient based on the target responses and the target journal entries.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a block diagram illustrating an exemplary computing environment, according to example embodiments.

FIG. 2 illustrates an example view of a graphical user interface presenting a clinician homepage accessible by clinician device, according to example embodiments.

FIG. 3A illustrates an example view of a graphical user interface presenting a patient's page accessible by clinician device, according to example embodiments.

FIG. 3B is a continuation of GUI illustrated in FIG. 3A for presenting a patient's page accessible by clinician device, according to example embodiments.

FIG. 3C is a continuation of GUI illustrated in FIG. 3A for presenting a patient's page accessible by clinician device, according to example embodiments.

FIG. 4A illustrates an example view of a graphical user interface presenting a patient's homepage accessible by patient device, according to example embodiments.

FIG. 4B illustrates an example view of a graphical user interface presenting a “My Mood” page accessible by patient device, according to example embodiments.

FIG. 4C illustrates an example view of a graphical user interface presenting a “Journal Entry” page accessible by patient device, according to example embodiments.

FIG. 4D illustrates an example view of a graphical user interface presenting an “Insights” page accessible by patient device, according to example embodiments.

FIG. 4E illustrates an example view of a graphical user interface presenting a “Library” page accessible by patient device, according to example embodiments.

FIG. 4F illustrates an example view of a graphical user interface presenting a “Journaling” page accessible by patient device, according to example embodiments.

FIG. 4G illustrates an example view a graphical user interface presenting a “Need Help” page accessible by patient device, according to example embodiments.

FIG. 5 is a block diagram illustrating operations associated with onboarding a patient and generating a health score of the patient, according to example embodiments.

FIG. 6 is a block diagram illustrating operations associated with onboarding a patient and generating a health score of the patient, according to example embodiments.

FIG. 7 is a flow diagram illustrating a method of generating a trained machine learning model, according to example embodiments.

FIG. 8 is a flow diagram illustrating a method of generating a health score for a patient, according to example embodiments.

FIG. 9A illustrates a system bus computing system architecture, according to example embodiments.

FIG. 9B illustrates a computer system having a chipset architecture that may represent at least a portion of the organization's computing system, according to example embodiments.

FIG. 10A illustrates an example view of a graphical user interface presenting a “Learn” page accessible by patient device, according to example embodiments.

FIG. 10B illustrates an example view of a graphical user interface presenting a “Path” page accessible by patient device, according to example embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

One or more techniques described herein provide a platform for matching patients with the proper therapists for mental health support and a suite of tools to complement the patient's therapy services. For example, the one or more techniques described herein leverage a unique machine learning system through which the platform is able to detect a patient's symptoms and provide a targeted system to therapy. Unlike conventional approaches to evaluating a patient's mental health through various machine learning approaches, the one or more techniques described herein include a training process by which the machine learning architecture is trained to mimic a clinician analyzing a patient's interactions. For example, the machine learning architecture may be configured to build a time series construct of patient feedback and analyze that time series construct to determine an overall health score of the patient. In this manner, the machine learning model provided herein is not limited to analyzing a single journal entry or a single questionnaire response; instead, the machine learning model is specifically constructed to analyze feedback from the patient in the context of the patient's previous submissions. Conventional mental health applications are simply unable to replicate this functionality.

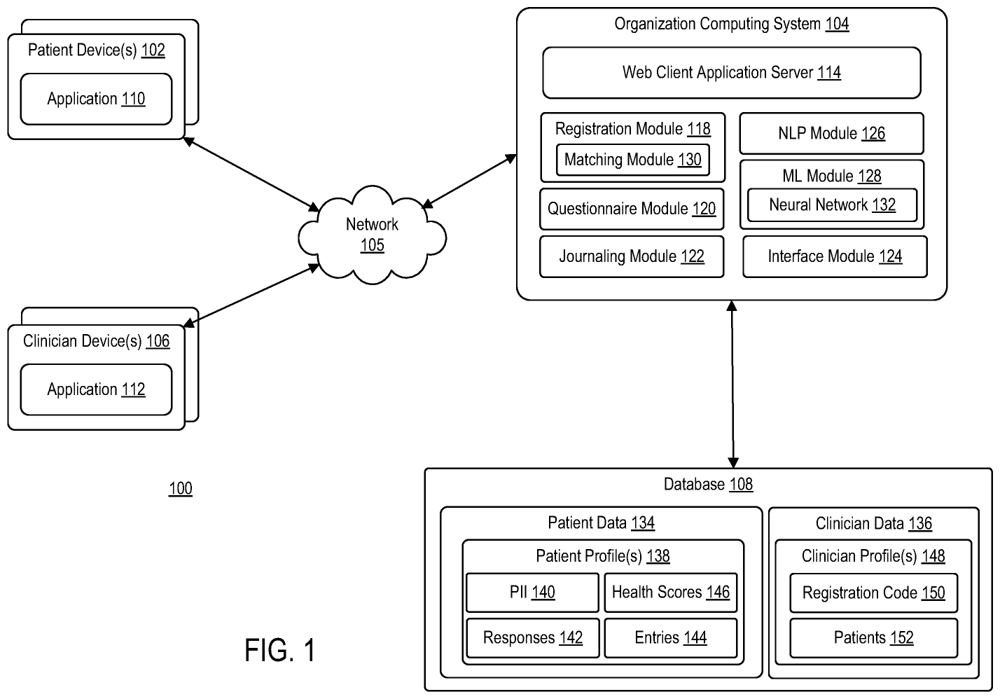

FIG. 1 is a block diagram illustrating a computing environment 100, according to example embodiments. Computing environment 100 may include one or more patient devices 102, one or more clinician devices 106, an organization computing system 104, and a database 108 communicating via one or more networks 105.

Network 105 may be of any suitable type, including individual connections via the Internet, such as cellular or Wi-Fi networks. In some embodiments, network 105 may connect terminals, services, and mobile devices using direct communications, such as radio frequencies identification (RFID), near-field communication (NFC), Bluetooth™, low-energy Bluetooth™ (BLE), Wi-Fi™, ZigBee™, ambient backscatter communication (ABC) protocols, universal serial bus (USB), wide area network (WAN), local area network (LAN), and the like. Because the information transmitted may be personal or confidential, security concerns may dictate that one or more of these types of connections be encrypted or otherwise secured. In some embodiments, however, the information being transmitted may be less personal, and therefore, the network connections may be selected for convenience over security.

Network 105 may include any type of computer networking arrangement used to exchange data or information. For example, network 105 may be representative of the Internet, a private data network, virtual private network, and/or other suitable connection(s) that enables components in computing environment 100 to send and receive information between the components of computing environment 100.

Patient device 102 may be operated by a patient. For example, patient device 102 may be representative of a mobile device, a tablet, a desktop computer, or any computing system having one or more of the capabilities described herein. Patient device 102 may include at least application 110. Application 110 may be representative of a web browser that allows access to a website or a stand-alone application. Patient device 102 may access application 110 to access functionality of organization computing system 104. Patient device 102 may communicate over network 105 to request a webpage or other information, for example, from web client application server 114 of organization computing system 104. For example, patient device 102 may be configured to execute application 110 to access one or more functionalities of organization computing system 104. The content that may be displayed to patient device 102 may be transmitted from web client application server 114 to patient device 102, and subsequently processed by application 110 for display through a display associated with patient device 102.

Clinician device 106 may be operated by a clinician. For example, clinician device 106 may be representative of a mobile device, a tablet, a desktop computer, or any computing system having one or more capabilities described herein. Clinician device 106 may include at least application 112. Application 112 may be representative of a web browser that allows access to a website or stand-alone application. Clinician device 106 may access application 112 to access functionality of organization computing system 104. Clinician device 106 may communicate over network 105 to request a webpage or other information, for example, from web client application server 114 of organization computing system 104. For example, clinician device 106 may be configured to execute application 112 to access one or more functionalities of organization computing system 104. The content that may be displayed to clinician device 106, and subsequently processed by application 112 for display through a display associated with clinician device 106.

Organization computing system 104 may be representative of one or more computer systems associated with an organization. Organization computing system 104 may include web client application server 114, registration module 118, questionnaire module 120, journaling module 122, interface module 124, NLP module 126, and machine learning (ML) module 128. Each registration module 118, questionnaire module 120, journaling module 122, interface module 124, NLP module 126, and ML module 128 may be formed from one or more software modules. The one or more software modules may be collections of instructions stored on a media (e.g., memory associated with organization computing system 104) that represents a series of machine instructions (e.g., program code) that implements one or more algorithmic steps. Such machine instructions may be the actual computer code a processor associated with organization computing system 104 interprets to implement the instructions, or, alternatively, may be a higher-level coding of the instructions that are interpreted to obtain the actual computer code. The one or more software modules may also include one or more hardware components. One or more aspects of the algorithm may be performed by the hardware components (e.g., circuitry) itself, rather as a result of an instruction.

Registration module 118 may be configured to register one or more of a clinician device 106 and/or a patient device 102 with organization computing system 104. In operation, clinician device 106 may register a profile with organization computing system 104 via application 110 executing thereon. In some embodiments, in response to a registration request from clinician device 106, registration module 118 may generate a clinician profile corresponding to the clinician associated with clinician device 106. In some embodiments, generating a clinician profile may include registration module 118 assigning the clinician a unique registration code. The unique registration code may be used by clinician device 106 for registering patients with the clinician corresponding to the unique registration code. For example, in operation, a clinician could share his or her unique registration code with a target patient for registration with organization computing system 104.

Further, patient device 102 may establish a profile with organization computing system 104 via application 112 executing thereon. In some embodiments, patient device 102 may establish a profile with organization computing system 104 using a unique registration code. For example, when signing up for an account with organization computing system 104, patient device 102 may provide registration module 118 with a unique registration code from a referring clinician. In some embodiments, in response to a registration request from patient device 102, registration module 118 may generate a patient profile corresponding to the patient associated with patient device 102.

In some embodiments, registration module 118 may include matching module 130. Matching module 130 may be configured to match a patient with a clinician registered with organization computing system 104. For example, as previously discussed, in some embodiments, a patient may register with organization computing system 104 using a unique registration code associated with a particular clinician. In this manner, the patient may be assigned the clinician associated with the unique registration code. As those skilled in the art recognize, however, in some embodiments, a patient may not have a unique registration code. In other words, the patient may not initially be associated with a target clinician. In such embodiments, matching module 130 may assist the patient in identifying a clinician that best fits the needs of the patient. For example, during the registration processing, matching module 130 may provide patient device 102 with a series of questions that attempts to match the patient with a clinician. Exemplary questions may include, but are not limited to, patient's personality and what they are looking for in therapist characteristics. These questions will be combined with traditional questions (e.g., zip code, distance willing to travel, age, sex, insurance, and the like). Using the patient's responses, matching module 130 may identify at least one clinician that could best serve the needs of the patient. Upon receiving a confirmation from patient device 102, registration module 118 may associate the patient with the identified clinician.

Questionnaire module 120 may be configured to manage one or more questionnaires submitted by patient device 102. In some embodiments, questionnaire module 120 may prompt patient device 102 periodically (e.g., daily) to fill out various questionnaires directed to assessing the mental health of the patient. In some embodiments, questionnaire module 120 may prompt patient device 102 by pushing notifications to patient device 102 for display thereon. In some embodiments, exemplary questionnaires may include patient health questionnaire-8 (“PHQ-8”), general anxiety disorder-7 (GAD-7) questionnaire, and the like. Via the daily or periodic questionnaires, organization computing system 104 may be configured to gather daily or periodic updates related to a patient's mental health.

Journaling module 122 may be configured to manage one or more journal entries submitted by patient device 102. In some embodiments, journaling module 122 may prompt patient device 102 periodically (e.g., daily) to submit a journal entry. In some embodiments, journaling module 122 may prompt patient device 102 periodically (e.g., daily) to submit a journal entry focused on a particular aspect of the patient's life (e.g., work, family, personal, health, etc.). In some embodiments, journaling module 122 may prompt patient device 102 to provide journal entries via one or more possible formats.

In some embodiments, patient device 102 may facilitate submission of a journal entry by presenting a user interface, via application 110, with an input field that allows a user to enter text. In some embodiments, patient device 102 may facilitate submission of a journal entry by granting application 110 access to a microphone device of patient device 102 so that a user may record the journal entry. In some embodiments, patient device 102 may facilitate submission of a journal entry by granting application 110 access to a camera device so that a patient may capture a picture of a written journal entry for uploading to organization computing system 104. Via the journaling functionality, organization computing system 104 may be configured to gather daily or periodic updates related to a patient's mental health.

NLP module 126 may be configured to analyze journaling module 122 to detect a semantic tone and/or sentiment reflected in a journal entry. NLP module 126 may scan a journal entry, upon uploading, to learn and understand the content contained therein. In some embodiments, such as when a journal entry is uploaded as a picture, NLP module 126 may perform one or more optical character recognition (OCR) techniques in order to properly analyze the contents contained therein. In some embodiments, NLP module 126 may detect a semantic tone and/or sentiment reflected in a journal entry on a sentence-by-sentence or line-by-line basis. In some embodiments, NLP module 126 may detect a semantic tone and/or sentiment reflected in a journal entry broadly across the entire journal entry.

ML module 128 may be configured to generate a health score (e.g., ROSE score) for a patient based on the patient's responses to questionnaires and submitted journal entries. For example, ML module 128 may be trained to mimic a clinician by building a time series construct of patient feedback, and analyzing the time series construct to determine an overall health score of the patient. In this manner, ML module 128 is not limited to analyzing a single journal entry or a single questionnaire response; instead, ML module 128 may be trained to analyze feedback from the patient in the context of the patient's previous submissions. As such, ML module 128 may generate a health score for the patient that takes into consideration a current journal entry, previous journal entries, and patient responses to questionnaires.

In some embodiments, ML module 128 may include a neural network 132 or, more generally, a machine learning model capable of capturing nonlinear decision spaces. In operations, ML module 128 may train neural network 132 to output a health score by a patient using various forms of reinforcement learning. For example, ML module 128 may provide neural network 132 with a plurality of training data sets. In some embodiments, the training data sets may include sentences and/or paragraphs as modified by NLP module 126 (e.g., various tags injected into sentences and/or paragraphs to signal semantic tone or sentiment reflected therein) and answers to questionnaires. Using this training data set, generative neural network 132 may be trained to output the health score reflective of a current state of the patient's mental health. In some embodiments, the plurality of training data sets may further be encoded with annotations from clinicians. In this manner, neural network 132 may be trained to output a health score that is reflective of how a clinician analyzes health records of the patient.

In some embodiments, the training data set may further include speech recognition data. In some embodiments, the training data set may include facial recognition data. Such speech recognition and/or facial recognition data may be used in sentiment analysis for early detection of depression and mood disorder symptoms. For example, a roadmap for voice recognition may be generated for early detection of depression and mood disorder symptoms. In another example, a roadmap for video analytics on facial micro expressions may be generated for early detection of depression and mood disorder symptoms.

Further, in some embodiments, clinician device 106 may have access to outputs or predictions from neural network 132. For example, given the clinician's expertise in evaluating a patient's mental health, clinician device 106 may be able to view a predicted output (e.g., health score) from neural network 132. For example, if neural network 132 outputs a certain health score that is inconsistent with a clinician's determination (e.g., a health score being indicative of depression), the clinician may be able to manually adjust the user's score. In operation, machine learning module 128 may use this adjustment to continually train neural network 132 so that during a re-analysis of the patient's responses, neural network 132 may generate a more accurate health score.

Interface module 124 may be configured to generate one or more graphical user interfaces (GUIs) for presentation on a display associated with one or more of patient device 102 and clinician device 106. In some embodiments, interface module 124 may be configured to generate one or more GUIs associated with application 110 and/or application 112. In some embodiments, interface module 124 may be configured to generate GUIs corresponding to one or more of a homepage for a patient, a journaling entry page for a patient, a questionnaire page for a patient, and the like. In some embodiments, interface module 124 may be configured to generate GUIs corresponding to one or more of a homepage for a clinician and/or a patient portal for the clinician.

As illustrated, in some embodiments, organization computing system 104 may be in communication with database 108. Database 108 may be configured to store various data associated with organization computing system 104. Database 108 may include, for example, patient data 134 and clinician data 136. Patient data 134 may include one or more patient profiles 138. Each patient profile 138 may include one or more of personal identification information 140, questionnaire responses 142, journal entries 144, and health scores 146. Personal identification information 140 may include various identification information associated with the patient. Exemplary personal identification information may include, but is not limited to, user name, password, address, age, gender, e-mail address, phone number, medical history, and the like. Questionnaire responses 142 may correspond to patient responses to questionnaires as recorded by questionnaire module 120. In some embodiments, each questionnaire response may include a date and time corresponding to submission of the patient's response. Journal entries 144 may correspond to one or more journal entries as recorded by journaling module 122. In some embodiments, each journal entry may include a date and time corresponding to submission of the patient's journal entry. Health scores 146 may be representative of one or more health scores generated by ML module 128. In some embodiments, each health score 146 may include a data and time corresponding to the generation thereof.

Clinician data 136 may include one or more clinician profiles 148. Each clinician profile 148 may include a clinician's unique registration code 150 and patients 152. Unique registration code 150 may be assigned by organization computing system 104. Unique registration code 150 may allow a patient to enroll with a given clinician. Patients 152 may correspond to each patient assigned to, or enrolled with, the clinician.

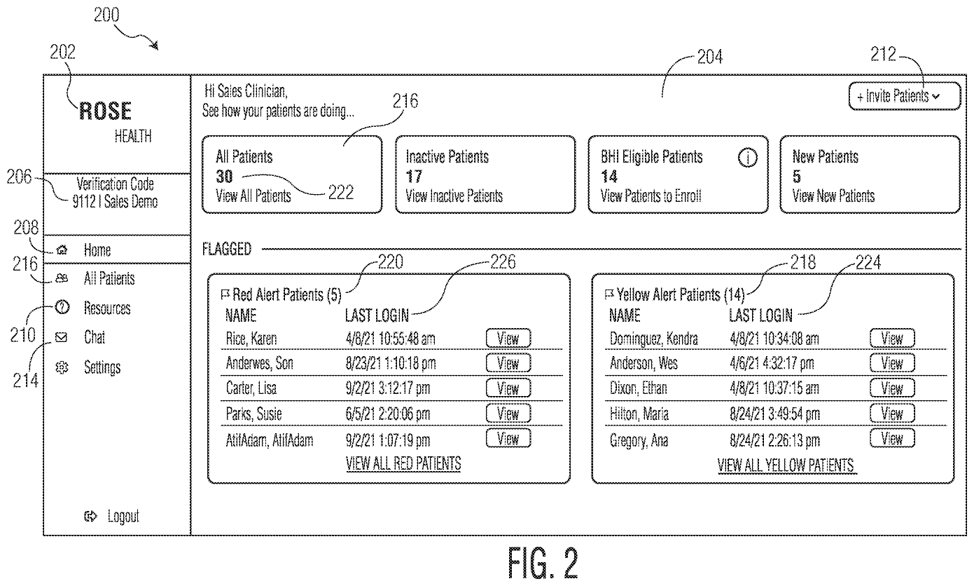

FIG. 2 illustrates an example view of a graphical user interface 200 presenting a clinician homepage accessible by clinician device 106, according to example embodiments. Graphical user interface (hereinafter “GUI 200”) may correspond to a GUI generated by interface module 124. In some embodiments, GUI 200 may be a web page presented in a web browser application (e.g., application 112) of clinician device 106. In some embodiments, GUI 200 may be a graphical user interface generated by a native software application (e.g., application 112) executing on clinician device 106.

As illustrated, GUI 200 may correspond to a homepage for a clinician. GUI 200 may include at least a first portion 202 and a second portion 204. First portion 202 may correspond to a navigation pane of GUI 200. First portion 202 may include one or more graphical elements 206-214 (e.g., buttons, links, text, etc.) selectable by the user to cause GUI 200 (e.g., the browser or native application) to send a message to organization computing system 104 requesting information associated with each graphical element 206-214.

Graphical element 206 may correspond to a unique registration code assigned to the clinician. The unique registration code may be used by clinician device 106 for registering patients with the clinician corresponding to the unique registration code. For example, in operation, a clinician could share his or her unique registration code with a target patient for registration with organization computing system 104.

Graphical element 208 may correspond to a homepage (e.g., the page currently shown in GUI 200). Accordingly, upon receiving input via graphical element 208, GUI 200 may request, from the organization computing system, navigation back to the homepage. Graphical element 210 may correspond to a resource tab. Upon interacting with the resource tab, a clinician may view informational and patient-focused marketing content. Graphical element 212 may correspond to an invite tab. Upon interacting with the invite tab, a clinician may send an invite to a target patient. In some embodiments, a clinician may send an invite to a target patient via email. In some embodiments, a clinician may send an invite to a target patient via text. The invite may include unique information corresponding to the clinician, such as, but not limited to, the unique registration code assigned to the clinician. Graphical element 214 may correspond to chat sessions. Upon interaction with chat session, a clinician may view chat messages sent from various patients. In some embodiments, chat sessions may include group messages with other clinical team members.

Second portion 204 may include one or more graphical elements 216-220 (e.g., buttons, links, text, etc.) selectable by the user to cause GUI 200 (e.g., the browser or native application) to send a message to organization computing system 104 requesting information associated with each graphical element 216-220.

Graphical element 216 may correspond to a listing of patients associated with the clinician. In some embodiments, graphical element 216 may include one or more sub-elements 222. Each sub-element 222 may correspond to a given patient. For example, as illustrated, sub-element 222 may correspond to “Patient #1.” In some embodiments, upon interaction with sub-element 222, clinician may be navigated to a page dedicated to “Patient #1.”

Graphical element 218 may correspond to a listing of patients that fall under a “Yellow Alert” category. For example, upon ML module 128 analyzing a patient's questionnaires and submitted journal entries to generate a health score for the patient, ML module 128 may determine that this health score falls within the range of a “Yellow Alert.” A “Yellow Alert” may correspond to those patients that may be concerning, but do not necessarily require review. In some embodiments, graphical element 218 may include one or more sub-elements 224. Each sub-element 224 may correspond to a given patient. For example, as illustrated, sub-element 224 may correspond to “Patient #2.” In some embodiments, upon interaction with sub-element 224, clinician may be navigated to a page dedicated to “Patient #2.”

Graphical element 220 may correspond to a listing of patients that fall under a “Red Alert” category. For example, upon ML module 128 analyzing a patient's questionnaires and submitted journal entries to generate a health score for the patient, ML module 128 may determine that this health score falls within the range of a “Red Alert.” A “Red Alert” may correspond to those patients that may be exhibiting potentially dangerous symptoms and require review. In some embodiments, graphical element 220 may include one or more sub-elements 226. Each sub-element 226 may correspond to a given patient. For example, as illustrated, sub-element 226 may correspond to “Patient #3.” In some embodiments, upon interaction with sub-element 226, clinician may be navigated to a page dedicated to “Patient #3.”

As those skilled in the art recognize, patients may be automatically moved among graphical elements 216-220, depending on a time series analysis of their respective journal entries by ML module 128. In this manner, graphical elements 216 and 218 may allow a clinician to timely identify and prioritize patients for intervention.

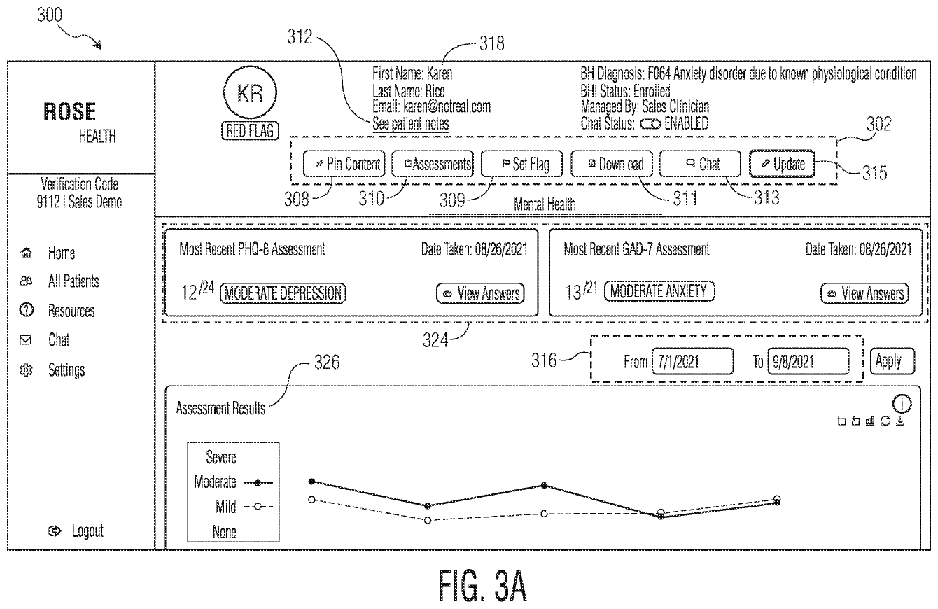

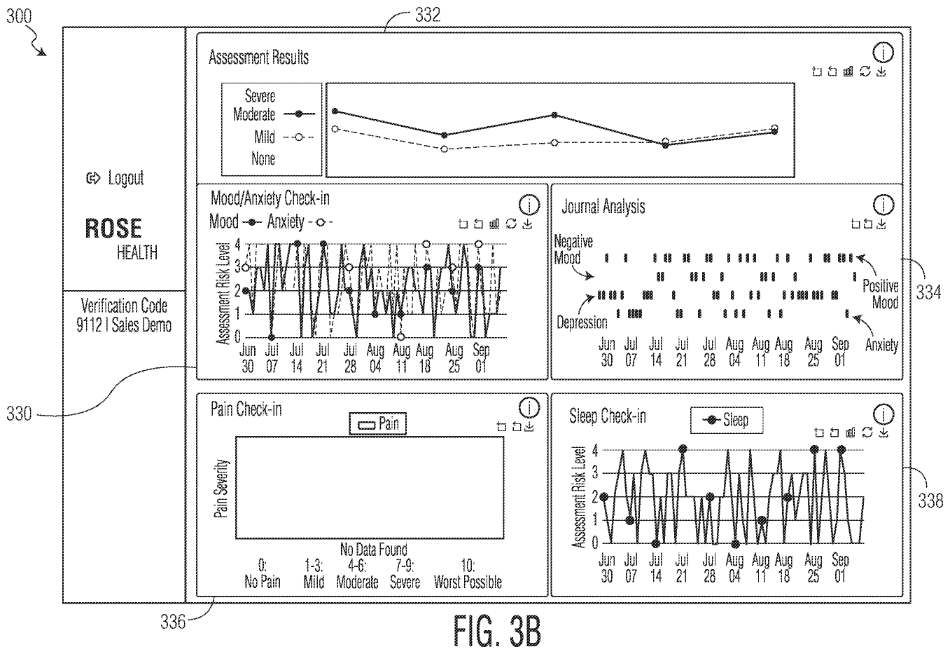

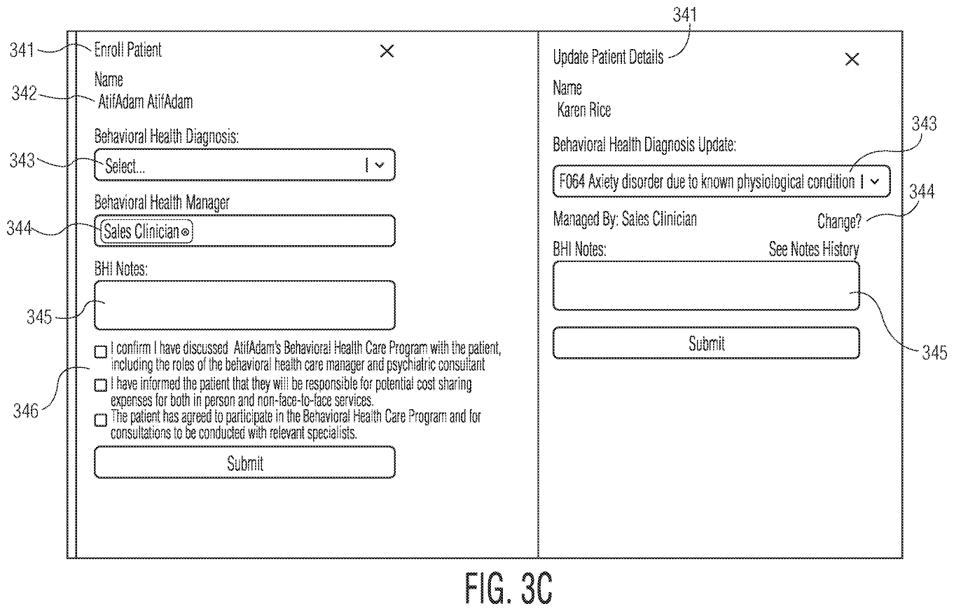

FIGS. 3A-3C illustrate an example view of a graphical user interface 300 presenting a patient's page accessible by clinician device 106, according to example embodiments. In some embodiments, FIGS. 3B and 3C may be extensions of FIG. 3A. Graphical user interface (hereinafter “GUI 300”) may correspond to a GUI generated by interface module 124. In some embodiments, GUI 300 may be a web page presented in a web browser application (e.g., application 112) of clinician device 106. In some embodiments, GUI 300 may be a graphical user interface generated by a native software application (e.g., application 112) executing on clinician device 106. In some embodiments, GUI 300 may be generated responsive to a clinician selecting any of sub-elements 222-226 corresponding to a respective patient.

As illustrated, GUI 300 may correspond to a page associated with a patient and accessible by the clinician via clinician device 106. Via GUI 300, clinician device 106 may access a given patient's data, such as, but not limited to, clinical assessments, mood tracking, questionnaire responses, journal entries, schedule appointments, and the like. Further, via GUI 300, clinician device 106 may pin or provide educational content to the patient.

GUI 300 may include toolbar 302. Via toolbar 302, a clinician may quickly access various functionality associated with organization computing system 104 for a given patient. Toolbar 302 may include one or more graphical elements 308, 309, 310, 311, 313, and 315 (e.g., buttons, links, text, etc.) selectable by the user to cause GUI 300 (e.g., the browser or native application) to send a message to organization computing system 104 requesting information associated with each graphical element 308, 309, 310, 311, 313, and 315.

Graphical element 308 may correspond to a pin content button. Via graphical element 308, a patient can select certain content to pin or mark for review for the patient. In some embodiments, the content may take the form of text content, audio content, video content, and the like. The patient can view such content via application 110 executing on patient device 102. Graphical element 310 may correspond to an assessment button. Via graphical element 310, a clinician can change the clinically validated assessments collected from the patient. In some embodiments, via graphical element 310, a clinician can also adjust the assessment. Graphical element 309 may allow the clinician to reset the patient alert. Via graphical element 309, a clinician may modify the patient's current flag (RED/YELLOW/NONE) and document their reasoning. Graphical element 311 may allow the clinician to download patients current chart. Via graphical element 311, a clinician may save a PDF of the patient's health report based on their desired date range. Graphical Element 313 may allow the clinician to review and send patient real-time messages. Via graphical element 313, a clinician may communicate with the patient through the Rose mobile app and send/receive text/picture messages. In some embodiments, via graphical element 313, clinician may also start an audio/video conversation with the patient.

Graphical element 312 may allow a user to view notes associated with a patient. For example, graphical element 312 may be a selectable link, which, when activated, provides the user with notes for the patient.

Graphical element 315 may correspond to allow clinician to document behavioral health management. Upon interacting with graphical element 315, a clinician is presented with management tab (e.g., graphical element 341). Graphical element 342 may allow a clinician to review patient name information. Graphical element 343 may allow clinician to pick relevant clinical diagnosis ICD-10 code from a drop down menu. Graphical element 344 allows clinician to add/change a clinical team member to the patient chart. Graphical element 345 may correspond to clinical notes. Via graphical element 345, a clinician may save comments or notes for the patient for future review. Graphical element 346 corresponds to patient consent documentation. Via 346, a clinician can document patient consent for behavioral health management.

As illustrated, GUI 300 may further include one or more sections 316, 318, 320, 324, and 326. Section 316 may correspond to the date range. Via section 316, a clinician may edit the date range to view all health information that may fall within a desired range. As illustrated, the present date range provided is Aug. 2, 2020 to Aug. 9, 2020.

Section 318 may correspond to a patient information section. Section 318 may include various information about the current patient. Such information may include a patient's unique identifier (UID), gender, date of birth (DOB), and issue. The UID may aid in keeping the patient's information confidential.

Section 320 may correspond to a journal entry's sections. Via section 320, a clinician may view a patient's journal entries. In some embodiments, each journal entry may include various information associated therewith. For example, as illustrated, each journal entry may include a date it was written, a semantic tone (as identified by NLP module 126, and a preview of the journal entry.

Section 324 may correspond to an assessments section. Via section 324, a clinician may view all generated assessments for the patient.

Section 326 may correspond to an assessment analysis section. Via section 326, a clinician may be able to see a health score generated for the patient by ML module 128. In some embodiments, the health score may include various information associated therewith. For example, as illustrated, each health score may include an overall assessment, a date the health score was generated, the score itself, an analysis of the score, and various answers to questionnaires provided by the patient.

GUI 300 may further include one or more sections 330-338.

Section 330 may correspond to a mood/anxiety chart. Via section 330 a clinician may be provided with a visualization of the patient's mood/anxiety levels of the patient over time.

Section 332 may correspond to a questionnaire chart. Via section 332 a clinician may be provided with a visualization of the patient's responses to the various questionnaire prompts (e.g., PHQ8/GAD7 prompts) over time.

Section 334 may correspond to a journal analysis. Via section 334 a clinician may be provided with a visualization of the patient's semantic tone levels, over time, as identified by NLP module 126.

Section 336 may correspond to an overall pain distribution chart. Via section 336, a clinician may be provided with a visualization of the patient's overall pain distribution over time. Section 338 may correspond to an overall sleep distribution chart. Via section 336, a clinician may be provided with a visualization of the patient's overall sleep distribution over time.

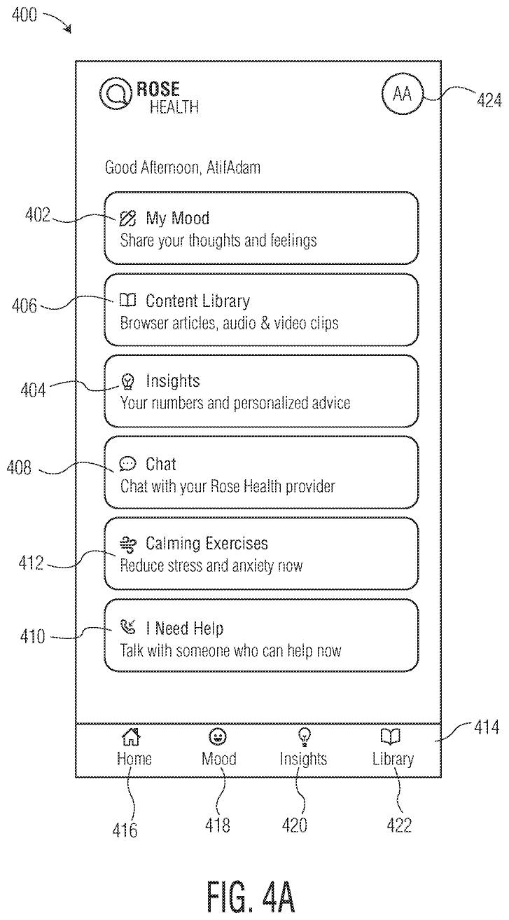

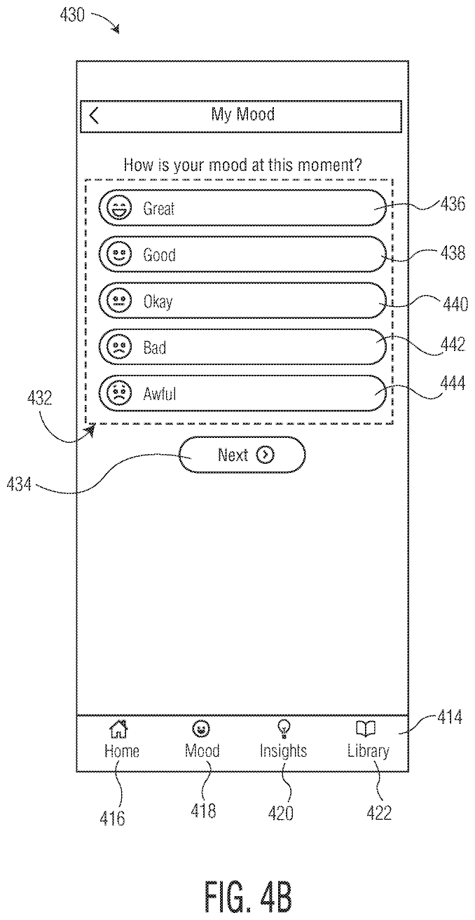

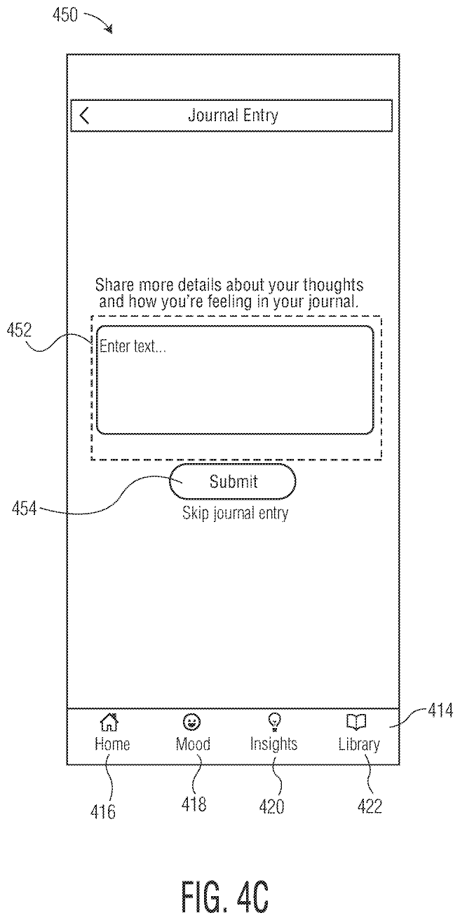

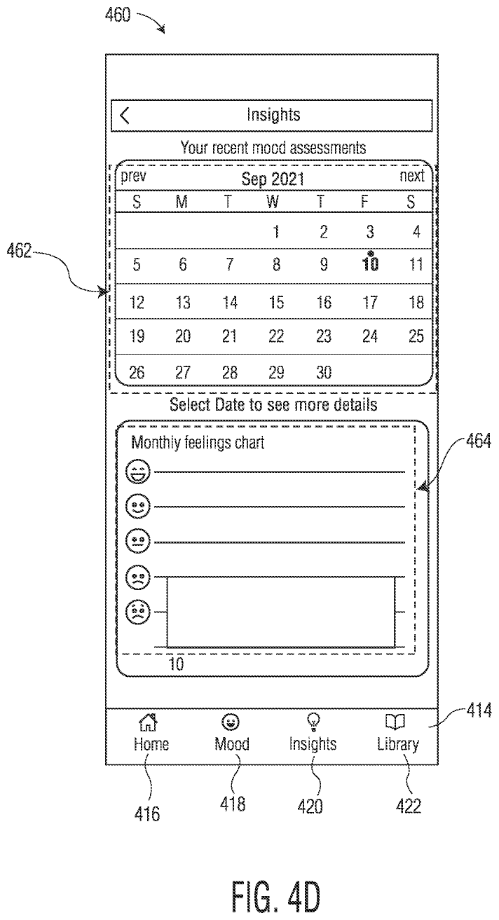

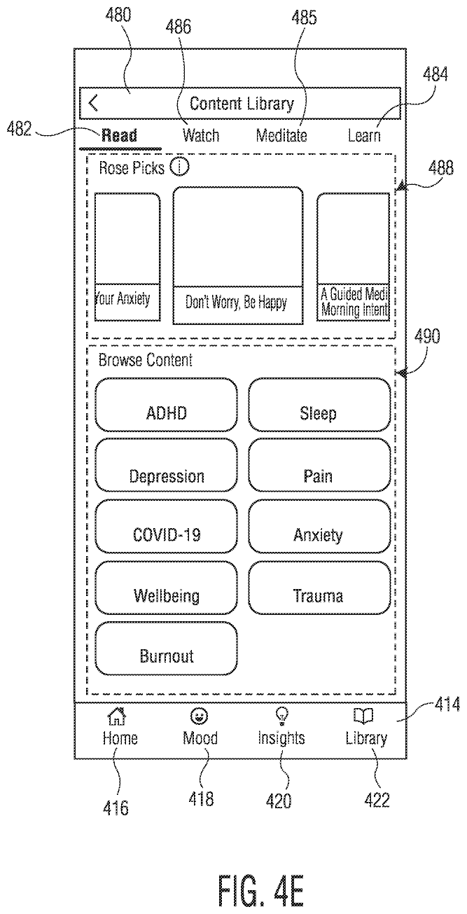

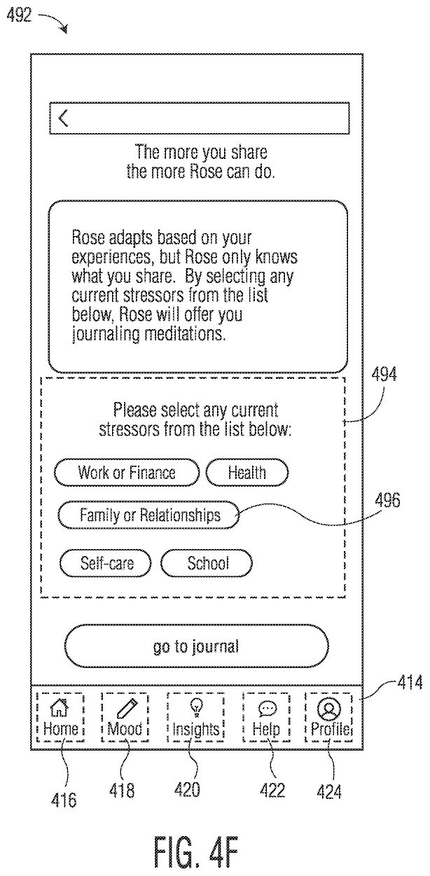

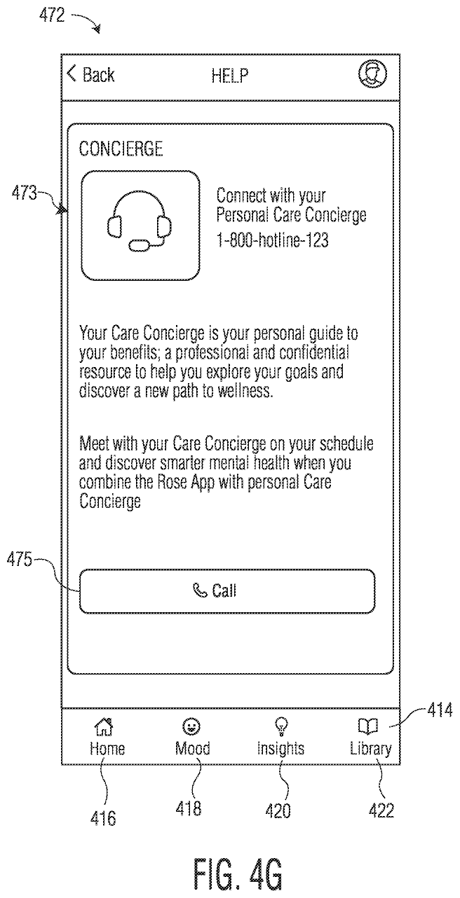

FIG. 4A illustrates an example view of a graphical user interface 400 presenting a patient's homepage accessible by patient device 102, according to example embodiments. Graphical user interface (hereinafter “GUI 400”) may correspond to a GUI generated by interface module 124. In some embodiments, GUI 400 may be a web page presented in a web browser application (e.g., application 110) of patient device 102. In some embodiments, GUI 400 may be a graphical user interface generated by a native software application (e.g., application 110) executing on patient device 102

As illustrated, GUI 400 may correspond to a page associated with a patient when the patient logs on to his or her account. GUI 400 may include one or more graphical elements 402-412 (e.g., buttons, links, text, etc.) selectable by the user to cause GUI 400 (e.g., the browser or native application) to send a message to organization computing system 104 requesting information associated with each graphical element 402-412.

Graphical element 402 may correspond to “My Mood.” When a patient selects graphical element 402, a patient may be provided with a subsequent GUI that allows the patient to provide feedback related to their mood and/or feeling. For example, via graphical element 402, a patient may provide feedback related to their mood and/or feeling via a PHQ-8 test.

Graphical element 404 may correspond to “Insights.” When a patient selects graphical element 404, a patient may be provided with various data related to their health score and/or personalized advice provided by the patient's clinician.

Graphical element 406 may correspond to “Content Library.” When a patient selects graphical element 406, patient may be provided with content related to various health issues. Exemplary content may include, but is not limited to, articles, audio clips, video clips, and the like.

Graphical element 408 may correspond to “Calming Exercises.” When a patient selects graphical element 408, the patient may be navigated pre-recorded meditation and breathing exercises. In some embodiments, the calming exercises may be a live video meditation sessions.

Graphical element 410 may correspond to “Need Help.” When a patient selects graphical element 410, the patient may be connected with an individual that can assist the patient. In some embodiments, graphical element 410 may be selected during an emergency, such as when the patient is experiencing a mental health emergency.

Graphical element 412 may correspond to “Chat.” When a patient selects graphical element 412, the patient message the provider directly through the app. In some embodiments, the chat session may be a video conference. In some embodiments, the chat session may be a phone conference.

In some embodiments, GUI 400 may further include navigation pane 414. Navigation pane 414 may allow a patient to quickly navigate to assorted pages within application 110. As illustrated, navigation pane 414 may include one or more graphical elements 416-422 (e.g., buttons, links, text, etc.) selectable by the user to cause GUI 400 (e.g., the browser or native application) to send a message to organization computing system 104 requesting information associated with each graphical element 416-422.

Graphical element 416 may correspond to a home button. When a patient selects graphical element 416, the patient may be navigated to the home page of application 110. Graphical element 418 may correspond to a mood button. When a patient selects graphical element 418, the patient may be navigated to a “My Mood” page. In some embodiments, interaction with graphical element 418 may navigate the patient to the same page as interaction with graphical element 402. Graphical element 420 may correspond to an insights button. When a patient selects graphical element 420, the patient may be navigated to an “Insights” page. In some embodiments, interaction with graphical element 420 may navigate the patient to the same page as interaction with graphical element 404. Graphical element 422 may correspond to a Library button. When a patient selects graphical element 422, the patient may be navigated to a Content Library page. Graphical element 424 may correspond to a profile button. When a patient selects graphical element 424, the patient may be navigated to their profile page.

FIG. 4G illustrates an example view of the graphical user interface (GUI) 472 presenting a “Need Help” page accessible by patient device 102 in when patient selects graphical element 410 from home screen. Graphical user interface (hereinafter “GUI 472”) may correspond to a GUI generated by interface module 124. In some embodiments, GUI 472 may be a web page presented in a web browser application (e.g., application 110) of patient device 102. In some embodiments, GUI 472 may be a graphical user interface generated by a native software application (e.g., application 110) executing on patient device 102. In some embodiments, GUI 472 may be generated responsive to a clinician selecting any of graphical element 410.

GUI 472 may include one or more graphical elements 473 and 475. Graphical element 473 may correspond a description of the in-app mental health help line service. Via graphical element 475, a patient may be able to call and speak with a “Rose Concierge” individual that can assist the patient. In some embodiments, graphical element 475 may be selected during an emergency, such as when the patient is experiencing a mental health emergency.

FIG. 4B illustrates an example view of a graphical user interface 430 presenting a “My Mood” page accessible by patient device 102, according to example embodiments. Graphical user interface (hereinafter “GUI 430”) may correspond to a GUI generated by interface module 124. In some embodiments, GUI 430 may be a web page presented in a web browser application (e.g., application 110) of patient device 102. In some embodiments, GUI 430 may be a graphical user interface generated by a native software application (e.g., application 110) executing on patient device 102. In some embodiments, GUI 430 may be generated responsive to a clinician selecting any of graphical element 402 or graphical element 418.

As illustrated, GUI 430 may correspond to a “My Mood” page. GUI 430 may include a set of graphical elements 432 and journal button 434. Set of graphical elements 432 may include one or more sub-elements 436-444. Each of sub-element 436-444 may correspond to a user's current mood. For example, sub-element 436 may correspond to a “great” mood; sub-element 438 may correspond to a “good” mood; sub-element 440 may correspond to an “okay” mood; sub-element 442 may correspond to a “bad” mood; sub-element 444 may correspond to an “awful” mood. Selection of any one or more sub-elements 436-444 may provide feedback to organization computing system 104 for determination of a health score for the patient.

Journal button 434 may correspond to a journal page. For example, after a patient provides feedback related to his or her health using a set of graphical elements 432, the patient may interact with journal button 434. Upon interaction with journal button 434, the patient may be navigated to a journal page.

FIG. 4C illustrates an example view of a graphical user interface 450 presenting a “Journal Entry” page accessible by patient device 102, according to example embodiments. Graphical user interface (hereinafter “GUI 450”) may correspond to a GUI generated by interface module 124. In some embodiments, GUI 450 may be a web page presented in a web browser application (e.g., application 110) of patient device 102. In some embodiments, GUI 450 may be a graphical user interface generated by a native software application (e.g., application 110) executing on patient device 102. In some embodiments, GUI 450 may be generated responsive to a patient selecting journal button 434.

GUI 450 may include text box 452 and graphical element 454. Via text box 452, a patient may provide input relating to how he or she is currently feeling. For example, via text box 452, a patient can submit a journal entry for processing by organization computing system 104. Based on the journal entry provided in text box 452 and feedback via a set of graphical elements 432, organization computing system 104 may be able to continually update the health score of the patient. Graphical element 454 may correspond to a submission button. For example, when a patient interacts with graphical element 454, patient device 102 may transmit the journal entry to organization computing system 104 for processing. Journal entries may be used for continually updating a health score of the patient.

In some embodiments, prior to be presented with GUI 450, a user may be presented with GUI 492. FIG. 4F illustrates an example view of a graphical user interface 492 presenting a journaling prompt, according to example embodiments. Graphical user interface (hereinafter “GUI 492”) may correspond to a GUI generated by interface module 124. In some embodiments, GUI 492 may be a web page presented in a web browser application (e.g., application 110) of patient device 102. In some embodiments, GUI 492 may be a graphical user interface generated by a native software application (e.g., application 110) executing on patient device 102. In some embodiments, GUI 492 may be generated responsive to a patient selecting journal button 434.

As shown, GUI 492 may include interactive portion 494. Interactive portion 494 may provide a user with various prompts to select a “stressor” they are currently experiencing. For example, interactive portion 494 may include one or more prompts 496, each prompt corresponding to a specific stressor. In this manner, before being provided with GUI 450 to submit a journal entry, a user may provide context to his or her journal entry.

FIG. 4D illustrates an example view of a graphical user interface 460 presenting an “Insights” page accessible by patient device 102, according to example embodiments. Graphical user interface (hereinafter “GUI 460”) may correspond to a GUI generated by interface module 124. In some embodiments, GUI 460 may be a web page presented in a web browser application (e.g., application 110) of patient device 102. In some embodiments, GUI 460 may be a graphical user interface generated by native software application (e.g., application 110) executing on patient device 102. In some embodiments, GUI 460 may be generated in response to a patient selecting one or more of graphical element 404 or graphical element 420.

GUI 460 may include one or more graphical elements 462-464. Graphical element 462 may correspond to a calendar. Via graphical element 462, a patient may access his or her recent mood assessments on a daily, weekly, or monthly basis. For example, via graphical element 462, a patient may be able to select a day to view a patient's mood assessment as of that day. In some embodiments, a patient may be able to select a day to view a patient's health score as of that day. In this manner, a patient may be able to keep track of his or her health score development over time.

Graphical element 464 may correspond to a visual representation of the patient's current average mood. For example, graphical element 464 may display a health score corresponding to a patient. For example, when a patient hovers over the marker, GUI 460 may be updated with an overlay window that provides the patient's current health score. In some embodiments, GUI 460 may include a sliding scale ranging from great to awful. Via the sliding scale, the patient may be able to view where his or her current average mood falls, based on a marker. In some embodiments, GUI 460 may correspond to a journal entry selection. For example, via GUI 460, a patient may be able to view his or her journal entries over time (e.g., by selecting a date via graphical element 462).

FIG. 4E illustrates an example view of a graphical user interface 480 presenting a “Library” page accessible by patient device 102, according to example embodiments. Graphical user interface (hereinafter “GUI 480”) may correspond to a GUI generated by interface module 124. In some embodiments, GUI 480 may be a web page presented in a web browser application (e.g., application 110) of patient device 102. In some embodiments, GUI 480 may be a graphical user interface generated by native software application (e.g., application 110) executing on patient device 102. In some embodiments, GUI 480 may be generated in response to a patient selecting graphical element 406.

GUI 460 may include one or more graphical elements 482-486. Each graphical element 482-486 may correspond to a specific type of content available to the user. For example, as illustrated, graphical element 482 may correspond to “read,” i.e., text based content. Upon interaction with graphical element 482, GUI 480 may be updated to display text-based content for the patient. Graphical element 484 may correspond to “listen,” i.e., audio content. Upon interaction with graphical element 484, GUI 480 may be updated to display audio content for the patient. Graphical element 485 may correspond to “meditate,” i.e., meditation exercise content. Upon interaction with graphical element 485, GUI 480 may be updated to display meditation exercises for the patient. In some embodiments, graphical element 485 may correspond to live meditation session hosted via video at specific times. Graphical element 486 may correspond to “watch,” i.e., video content. Upon interaction with graphical element 486, GUI 480 may be updated to display video content for the patient. As illustrated in FIG. 4E, a patient has selected graphical element 482 corresponding to text-based content.

GUI 460 may further include one or more sections 488 and 490. Section 488 may correspond to content that is recommended to the patient. For example, section 488 may correspond to content that is recommended to the patient based on the patient's current health score. In some embodiments, section 488 may correspond to content that is recommended to the patient based on hand-selected content provided by the clinician of the patient.

Section 490 may correspond to all content available to the patient. As illustrated, section 490 may provide patients with the ability to browse content based on a category type. For example, the patient may be able to browse and/or view content corresponding to anxiety, depression, well- being, trauma, and the like.

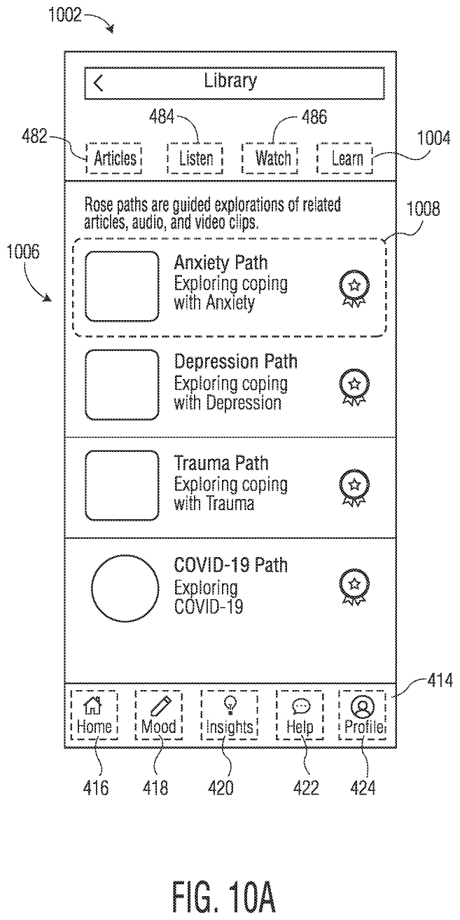

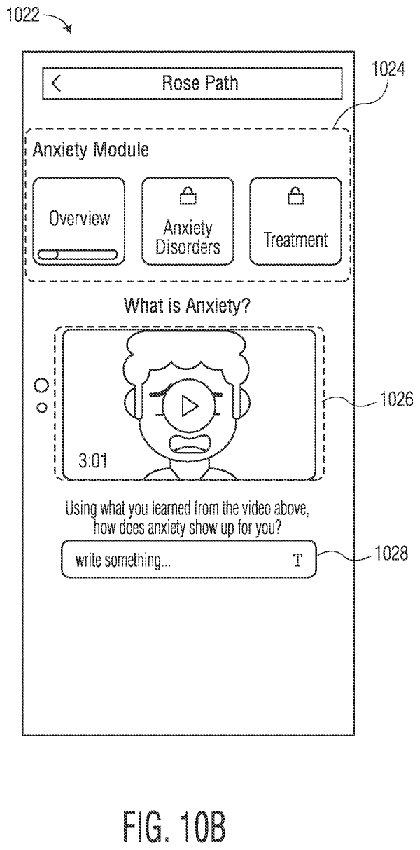

FIG. 10A illustrates an example view of a graphical user interface 1002 presenting a “Library” page accessible by patient device 102, according to example embodiments. Graphical user interface (hereinafter “GUI 1002”) may correspond to a GUI generated by interface module 124. In some embodiments, GUI 1002 may be a web page presented in a web browser application (e.g., application 110) of patient device 102. In some embodiments, GUI 1002 may be a graphical user interface generated by native software application (e.g., application 110) executing on patient device 102. In some embodiments, GUI 1002 may be generated in response to a patient selecting graphical element 406.

GUI 1002 may be similar to GUI 480. However, as shown, in addition to graphical elements 482-486, GUI 1002 may further include graphical element 1004. Graphical element 1004 may correspond to a “learn” tab. Via the learn tab, a user may be provided with various paths (e.g., Rose paths) that are guided explorations of related articles, audio, and video clips associated with certain health issues.

As shown, GUI 1002 may include section 1006. Section 1006 may include one or more paths 1008. Each path 1008 may correspond to a specific health issue. As shown, paths 1008 may include an anxiety path, a depression path, a trauma path, and a Covid-19 path.

FIG. 10B illustrates an example view of a graphical user interface 1022 presenting a “Rose Path” page accessible by patient device 102, according to example embodiments. Graphical user interface (hereinafter “GUI 1022”) may correspond to a GUI generated by interface module 124. In some embodiments, GUI 1022 may be a web page presented in a web browser application (e.g., application 110) of patient device 102. In some embodiments, GUI 1022 may be a graphical user interface generated by native software application (e.g., application 110) executing on patient device 102. In some embodiments, GUI 1022 may be generated in response to a patient selecting anxiety path 1008 in GUI 1002.

GUI 1022 may include module section 1024. Module section 1024 may include one or more selectable modules. For example, module section 1024 may show a user each module in the selected path. In this specific example, the user is provided with various module related to the anxiety path. Via module section 1024, a user can track his or her progress through the anxiety path.

GUI 1022 may further include multimedia 1026 related to the current module. For example, as shown, the user is currently in the overview module. Multimedia 1026 may take the form of a video explaining what anxiety is to the user.

Further, GUI 1022 may also include an interactive section 1028. Via interactive section 1028 a user may provide feedback related to multimedia 1026 that the user viewed. As shown, interactive section 1028 may prompt the user to describe what they learned from multimedia 1026.

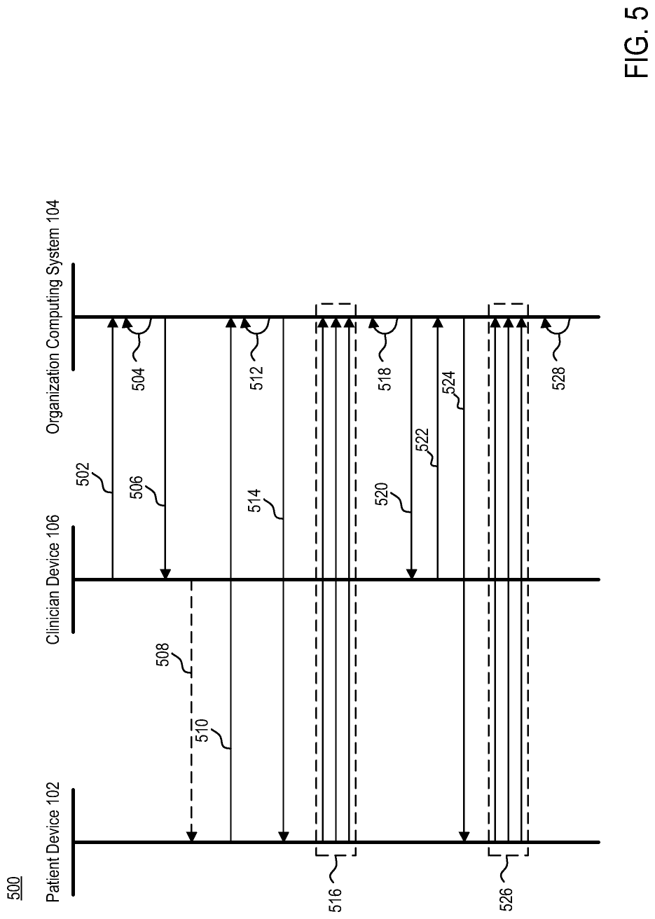

FIG. 5 is a block diagram 500 illustrating operations associated with onboarding a patient and generating a health score of the patient, according to example embodiments.

At operation 502, clinician device 106 may begin a registration process with organization computing system 104. In some embodiments, clinician device 106 may access organization computing system 104 by navigating to a web page via application 112 executing thereon. In some embodiments, clinician device 106 may access organization computing system 104 by downloading and installing a native application (e.g., application 112) associated with organization computing system 104. During the registration process, clinician device 106 may register a user name and/or email address uniquely associated with a clinician of clinician device 106.

At operation 504, organization computing system 104 may receive the registration request from clinician device 106. In some embodiments, in response to receiving the registration request, organization computing system 104 may establish a clinician profile for clinician device 106. For example, organization computing system 104 may update clinician data 136 in database 108 with a new clinician profile corresponding to clinician device 106. In some embodiments, in response to receiving the registration request, organization computing system 104 may assign to the clinician a unique registration code. The unique registration code may be used by clinician device 106 for registering patients with the clinician corresponding to the unique registration code. For example, in operation, a clinician could share his or her unique registration code with a target patient for registration with organization computing system 104. At operation 506, organization computing system 104 may provide the unique registration code to clinician device 106.

In some embodiments, at operation 508, clinician device 106 may share the unique registration code with a patient. For example, clinician device 106 may send a message (e.g., email, text, etc.) to patient device 102 with the unique registration code and instructions for registering a profile with organization computing system 104.

At operation 510, patient device 102 may begin a registration process with organization computing system 104. In some embodiments, patient device 102 may access organization computing system 104 by navigating to a web page via application 110 executing thereon. In some embodiments, patient device 102 may access organization computing system 104 by downloading and installing a native application (e.g., application 110) associated with organization computing system 104. During the registration process, patient device 102 may register a user name and/or email address uniquely associated with a patient associated with patient device 102. In some embodiments, as part of the registration process, patient device 102 may provide organization computing system 104 with the unique registration code provided by clinician device 106. In this manner, patient device 102 may instruct or notify organization computing system 104 to associate the patient with the target clinician.

At operation 512, organization computing system 104 may receive the registration request from patient device 102. In some embodiments, in response to receiving the registration request, organization computing system 104 may establish a patient profile for patient device 102. For example, organization computing system 104 may update patient data 134 in database 108 with a new patient profile corresponding to patient device 102. In some embodiments, in response to receiving a unique registration code from patient device 102 as part of the registration process, organization computing system 104 may associate the patient's profile with the target clinician's profile. In this manner, organization computing system 104 may enroll the patient as a patient for the target clinician. At operation 514, organization computing system 104 may notify patient device 102 that the profile is ready.

At operation 516, patient device 102 may interact with organization computing system 104 via application 110 executing thereon. In some embodiments, patient device 102 may interact with the organization computing system via one or more prompts provided by questionnaire module 120. For example, questionnaire module 120 may prompt patient device 102 periodically (e.g., daily, weekly, etc.) to fill out various questionnaires directed to assessing the mental health of the patient. In some embodiments, questionnaire module 120 may prompt patient device 102 by pushing notifications to patient device 102 for display thereon. In some embodiments, exemplary questionnaires may include PHQ-8, GAD-7 questionnaire, and the like.

In some embodiments, patient device 102 may interact with organization computing system 104 via one or more prompts provided by journaling module 122. In some embodiments, journaling module 122 may prompt patient device 102 periodically (e.g., daily, weekly, etc.) to submit a journal entry. In some embodiments, journaling module 122 may prompt patient device 102 periodically (e.g., daily) to submit a journal entry focused on a particular aspect of the patient's life (e.g., work, family, personal, health, etc.). In some embodiments, journaling module 122 may prompt patient device 102 to provide journal entries via one or more possible formats.

In some embodiments, patient device 102 may facilitate submission of a journal entry by presenting a user interface, via application 110, with an input field that allows a user to enter text. In some embodiments, patient device 102 may facilitate submission of a journal entry by granting application 110 access to a microphone device of patient device 102 so that a user may record the journal entry. In some embodiments, patient device 102 may facilitate submission of a journal entry by granting application 110 access to a camera device so that a patient may capture a picture of a written journal entry for uploading to organization computing system 104.

At operation 518, organization computing system 104 may process inputs received from patient device 102. In some embodiments, processing inputs received from patient device 102 may include NLP module 126 processing journal entries submitted by patient device 102. For example, NLP module 126 may analyze journal entries provided by patient device 102 to detect a semantic tone and/or sentiment reflected in a journal entry. In some embodiments, NLP module 126 may scan a journal entry, upon uploading, to learn and understand the content contained therein. In some embodiments, such as when a journal entry is uploaded as a picture, NLP module 126 may perform one or more optical character recognition (OCR) techniques in order to properly analyze the contents contained therein. In some embodiments, NLP module 126 may detect a semantic tone and/or sentiment reflected in a journal entry on a sentence-by-sentence or line-by- line basis. In some embodiments, NLP module 126 may detect a semantic tone and/or sentiment reflected in a journal entry broadly across the entire journal entry.

Further, processing inputs received from patient device 102 may include ML module 128 generating a health score (e.g., ROSE score) for a patient based on the patient's responses to questionnaires and submitted journal entries. For example, ML module 128 may be trained to mimic a clinician by building a time series construct of patient feedback, and analyzing the time series construct to determine an overall health score of the patient. In this manner, ML module 128 is not limited to analyzing a single journal entry or a single questionnaire response; instead, ML module 128 may be trained to analyze feedback from the patient in the context of the patient's previous submissions. As such, ML module 128 may generate a health score for the patient that takes into consideration a current journal entry, previous journal entries, and patient responses to questionnaires.

At operation 520, organization computing system 104 may make the patient's data accessible to clinician device 106. For example, organization computing system 104 may notify clinician device 106 that new or updated patient data is available for review and/or evaluation.

At operation 522, clinician device 106 may access the patient data. For example, clinician device 106 may access patient data via application 112 executing thereon. In some embodiments, accessing patient data may include clinician device 106 modifying a health score associated with the patient. For example, a clinician may use his or her expertise to modify the patient's health score (as generated by ML module 128) based on the clinician's knowledge of the patient through various sessions with the patient.

At operation 524, organization computing system 104 may provide the health score to patient device 102. For example, organization computing system 104 may notify patient device 102 whenever an updated health score is generated by ML module 128.

At operation 526, patient device 102 may continue to interact with organization computing system 104 via application 110 executing thereon. In some embodiments, patient device 102 may continue to interact with the organization computing system via one or more prompts provided by questionnaire module 120. For example, questionnaire module 120 may continue to prompt patient device 102 periodically (e.g., daily, weekly, etc.) to fill out various questionnaires directed to assessing the mental health of the patient. In some embodiments, patient device 102 may continue to interact with organization computing system 104 via one or more prompts provided by journaling module 122.

At operation 528, organization computing system 104 may continue to process inputs received from patient device 102. In some embodiments, processing inputs received from patient device 102 may include NLP module 126 processing journal entries submitted by patient device 102. For example, NLP module 126 may analyze journal entries provided by patient device 102 to detect a semantic tone and/or sentiment reflected in a journal entry. In some embodiments, continuing to process inputs received from patient device 102 may include ML module 128 may include continuing to generate updated health scores (e.g., ROSE score) for a patient based on the patient's responses to questionnaires and submitted journal entries.

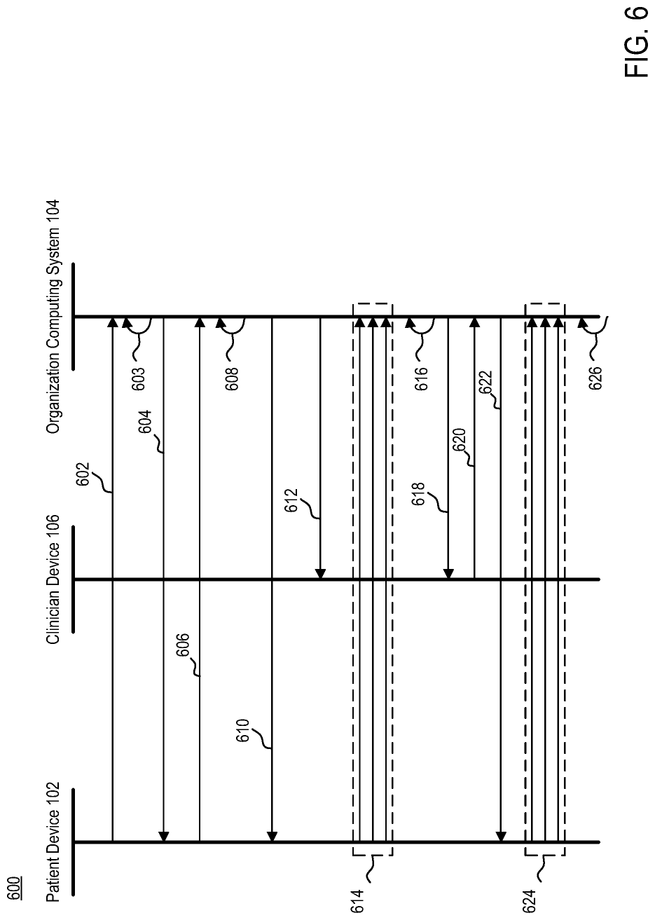

FIG. 6 is a block diagram 600 illustrating operations associated with onboarding a patient and generating a health score of the patient, according to example embodiments.

At operation 602, patient device 102 may begin a registration process with organization computing system 104. In some embodiments, patient device 102 may access organization computing system 104 by navigating to a web page via application 110 executing thereon. In some embodiments, patient device 102 may access organization computing system 104 by downloading and installing a native application (e.g., application 110) associated with organization computing system 104. During the registration process, patient device 102 may register a user name and/or email address uniquely associated with a patient of patient device 102.

At operation 603, organization computing system 104 may receive the registration request from patient device 102. In some embodiments, in response to receiving the registration request, organization computing system 104 may establish a patient profile for patient device 102. For example, organization computing system 104 may update patient data 134 in database 108 with a new patient profile corresponding to patient device 102. In some embodiments, registration of patient device 102 may include patient device 102 providing organization computing system 104 with a unique registration code corresponding to a clinician with which the patient wishes to register. However, in some embodiments, such as that described in FIG. 6, patient device 102 may not provide organization computing system 104 with a unique registration code. In such embodiments, organization computing system 104 may facilitate matching the patient with a clinician.

Matching module 130 may be configured to match a patient with a clinician registered with organization computing system 104. For example, matching module 130 may assist the patient in identifying a clinician that best fits the needs of the patient. To facilitate the matching process, at operation 604, matching module 130 may provide patient device 102 with a series of questions that attempts to match the patient with a clinician.

At operation 606, patient device 102 may receive the series of questions from organization computing system 104. Patient device 102 may transmit responses to each question in the series of questions to organization computing system 104 for processing.

At operation 608, organization computing system 104 may match the patient to a clinician. For example, using the patient's responses, matching module 130 may identify at least one clinician that could best serve the needs of the patient. Registration module 118 may associate the patient with the identified clinician. At operation 610, organization computing system 104 may notify patient device 102 of the matched clinician. At operation 612, organization computing system 104 may notify a respective clinician device 106 of the new patient.

At operation 614, patient device 102 may interact with organization computing system 104 via application 110 executing thereon. In some embodiments, patient device 102 may interact with the organization computing system via one or more prompts provided by questionnaire module 120. For example, questionnaire module 120 may prompt patient device 102 periodically (e.g., daily, weekly, etc.) to fill out various questionnaires directed to assessing the mental health of the patient. In some embodiments, questionnaire module 120 may prompt patient device 102 by pushing notifications to patient device 102 for display thereon. In some embodiments, exemplary questionnaires may include PHQ-8, GAD-7 questionnaire, and the like.

In some embodiments, patient device 102 may interact with organization computing system 104 via one or more prompts provided by journaling module 122. In some embodiments, journaling module 122 may prompt patient device 102 periodically (e.g., daily, weekly, etc.) to submit a journal entry. In some embodiments, journaling module 122 may prompt patient device 102 periodically (e.g., daily) to submit a journal entry focused on a particular aspect of the patient's life (e.g., work, family, personal, health, etc.). In some embodiments, journaling module 122 may prompt patient device 102 to provide journal entries via one or more possible formats.

In some embodiments, patient device 102 may facilitate submission of a journal entry by presenting a user interface, via application 110, with an input field that allows a user to enter text. In some embodiments, patient device 102 may facilitate submission of a journal entry by granting application 110 access to a microphone device of patient device 102 so that a user may record the journal entry. In some embodiments, patient device 102 may facilitate submission of a journal entry by granting application 110 access to a camera device so that a patient may capture a picture of a written journal entry for uploading to organization computing system 104.

At operation 616, organization computing system 104 may process inputs received from patient device 102. In some embodiments, processing inputs received from patient device 102 may include NLP module 126 processing journal entries submitted by patient device 102. For example, NLP module 126 may analyze journal entries provided by patient device 102 to detect a semantic tone and/or sentiment reflected in a journal entry. In some embodiments, NLP module 126 may scan a journal entry, upon uploading, to learn and understand the content contained therein. In some embodiments, such as when a journal entry is uploaded as a picture, NLP module 126 may perform one or more optical character recognition (OCR) techniques in order to properly analyze the contents contained therein. In some embodiments, NLP module 126 may detect a semantic tone and/or sentiment reflected in a journal entry on a sentence-by-sentence or line-by-line basis. In some embodiments, NLP module 126 may detect a semantic tone and/or sentiment reflected in a journal entry broadly across the entire journal entry.

Further, processing inputs received from patient device 102 may include ML module 128 generating a health score (e.g., ROSE score) for a patient based on the patient's responses to questionnaires and submitted journal entries. For example, ML module 128 may be trained to mimic a clinician by building a time series construct of patient feedback, and analyzing the time series construct to determine an overall health score of the patient. In this manner, ML module 128 is not limited to analyzing a single journal entry or a single questionnaire response; instead, ML module 128 may be trained to analyze feedback from the patient in the context of the patient's previous submissions. As such, ML module 128 may generate a health score for the patient that takes into consideration a current journal entry, previous journal entries, and patient responses to questionnaires.

At operation 618, organization computing system 104 may make the patient's data accessible to clinician device 106. For example, organization computing system 104 may notify clinician device 106 that new or updated patient data is available for review and/or evaluation.

At operation 620, clinician device 106 may access the patient data. For example, clinician device 106 may access patient data via application 112 executing thereon. In some embodiments, accessing patient data may include clinician device 106 modifying a health score associated with the patient. For example, a clinician may use his or her expertise to modify the patient's health score (as generated by ML module 128) based on the clinician's knowledge of the patient through various sessions with the patient.

At operation 622, organization computing system 104 may provide the health score to patient device 102. For example, organization computing system 104 may notify patient device 102 whenever an updated health score is generated by ML module 128.

At operation 624, patient device 102 may continue to interact with organization computing system 104 via application 110 executing thereon. In some embodiments, patient device 102 may continue to interact with the organization computing system via one or more prompts provided by questionnaire module 120. For example, questionnaire module 120 may continue to prompt patient device 102 periodically (e.g., daily, weekly, etc.) to fill out various questionnaires directed to assessing the mental health of the patient. In some embodiments, patient device 102 may continue to interact with organization computing system 104 via one or more prompts provided by journaling module 122.

At operation 626, organization computing system 104 may continue to process inputs received from patient device 102. In some embodiments, processing inputs received from patient device 102 may include NLP module 126 processing journal entries submitted by patient device 102. For example, NLP module 126 may analyze journal entries provided by patient device 102 to detect a semantic tone and/or sentiment reflected in a journal entry. In some embodiments, continuing to process inputs received from patient device 102 may include ML module 128 may include continuing to generate updated health scores (e.g., ROSE score) for a patient based on the patient's responses to questionnaires and submitted journal entries.

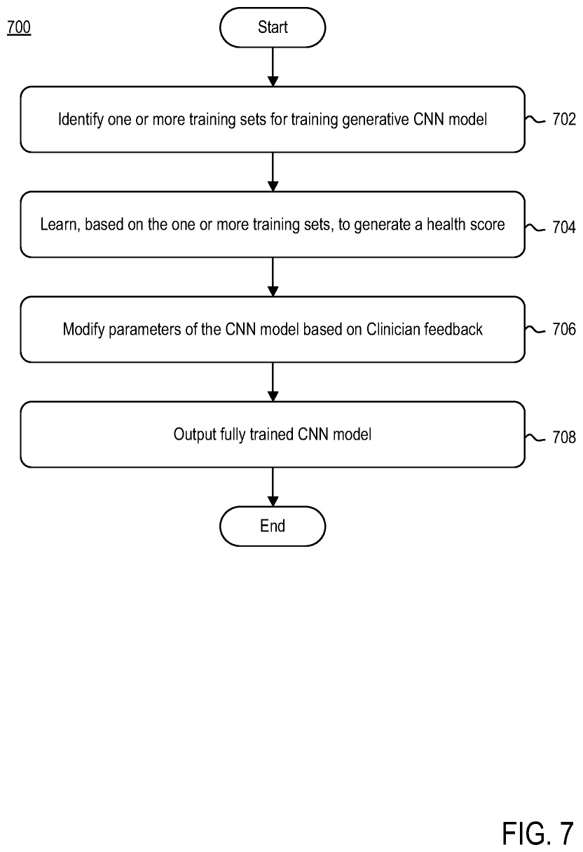

FIG. 7 is a flow diagram illustrating a method 700 of generating a trained machine learning model, according to example embodiments. Method 700 may begin at step 702.

At step 702, organization computing system 104 may identify one or more training data sets for training neural network 132. In some embodiments, the training data sets may include sentences and/or paragraphs as modified by NLP module 126 (e.g., various tags injected into sentences and/or paragraphs to signal semantic tone or sentiment reflected therein) and answers to questionnaires.

At step 704, neural network 132 may learn, based on the one or more training data sets, how to generate a health score for a patient. For example, ML module 128 may train neural network 132 to output a health score reflective of a current state of the patient's mental health. In some embodiments, the plurality of training data sets may further be encoded with annotations from clinicians. In this manner, neural network 132 may be trained to output a health score that is reflective of how a clinician analyzes health records of the patient.

At step 706, organization computing system 104 may modify parameters of neural network 132 based on clinician feedback. For example, during the training process, clinician device 106 may have access to the training results. Given the clinician's expertise in evaluating a patient's mental health, clinician device 106 may be able to view results of the training process in order to adjust weights or definitions used by neural network 132. For example, if neural network 132 maps a certain response from a patient as being indicative of depression, a clinician may be able to change the definition of depression or adjust the weights of neural network 132 so that during a re-analysis of the patient's response, neural network 132 may not associate the certain response with being indicative of depression.

At step 708, organization computing system 104 may output a fully trained neural network 132. For example, based on the training data sets and the modifications from clinician feedback, ML module 128 may implement a fully trained neural network 132 to generate health scores for patients based on patient interaction.

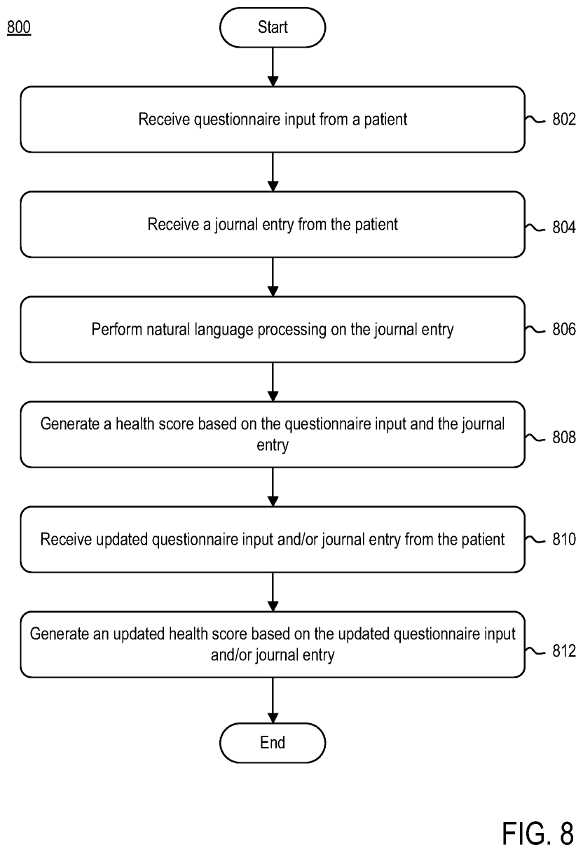

FIG. 8 is a flow diagram illustrating a method 800 of generating a health score for a patient, according to example embodiments. Method 800 may begin at step 802.

At step 802, organization computing system 104 may receive questionnaire input from patient device 102. For example, organization computing system 104 may receive questionnaire input from patient device 102 as a result of questionnaire module 120 prompting patient device 102 periodically (e.g., daily, weekly, etc.) to fill out various questionnaires directed to assessing the mental health of the patient. In some embodiments, exemplary questionnaire input may take the form of responses to various mental health questionnaires, such as, but not limited to, PHQ-8, GAD-7 questionnaire, and the like.

At step 804, organization computing system 104 may receive a journal entry from patient device 102. For example, organization computing system 104 may receive a journal entry as a result of one or more prompts provided by journaling module 122. In some embodiments, journaling module 122 may prompt patient device 102 periodically (e.g., daily, weekly, etc.) to submit a journal entry. In some embodiments, the journal entry received from patient device 102 may be focused on a particular aspect of the patient's life (e.g., work, family, personal, health, etc.). In some embodiments, organization computing system 104 may receive the journal entry via a graphical user interface presented via patient device 102. In some embodiments, the journal entry may be in the form of an audio file. In some embodiments, the journal entry may be in the form of a picture file (e.g., .jpeg, .tiff, .png, and the like).

At step 806, organization computing system 104 may perform natural language processing on the journal entry. NLP module 126 may process journal entries submitted by patient device 102. For example, NLP module 126 may analyze journal entries provided by patient device 102 to detect a semantic tone and/or sentiment reflected in a journal entry. In some embodiments, NLP module 126 may scan a journal entry, upon uploading, to learn and understand the content contained therein. In some embodiments, such as when a journal entry is uploaded as a picture, NLP module 126 may perform one or more OCR techniques in order to properly analyze the contents contained therein. In some embodiments, NLP module 126 may detect a semantic tone and/or sentiment reflected in a journal entry on a sentence-by-sentence or line-by-line basis. In some embodiments, NLP module 126 may detect a semantic tone and/or sentiment reflected in a journal entry broadly across the entire journal entry.

At step 808, organization computing system 104 may generate a health score for the patient based on one or more of the questionnaire input and/or the journal entry. ML module 128 may provide the questionnaire input and/or the journal entry to the fully trained neural network 132 for analysis. Neural network 132 may be trained to mimic a clinician by building a time series construct of patient feedback, and analyzing the time series construct to determine an overall health score of the patient. As output, neural network 132 may provide a health score for the patient.

At step 810, organization computing system 104 may receive updated questionnaire input and/or journal entry from patient device 102. For example, organization computing system 104 may receive updated questionnaire input and/or journal entry from patient device 102 in accordance with the functionality discussed above in conjunction with steps 802 and 804, respectively.

At step 812, organization computing system 104 may generate an updated health score based on the updated questionnaire input and/or journal entry from patient device 102. As previously discussed, ML module 128 is not limited to analyzing a single journal entry or a single questionnaire response. Instead, neural network 132 may be trained to analyze feedback from the patient in the context of the patient's previous submissions. As such, neural network 132 may generate a health score for the patient that takes into consideration a current journal entry, previous journal entries, and patient responses to questionnaires. In this manner, ML module 128 may build a time series construct of patient feedback, and analyze the time series construct to determine an overall health score of the patient.

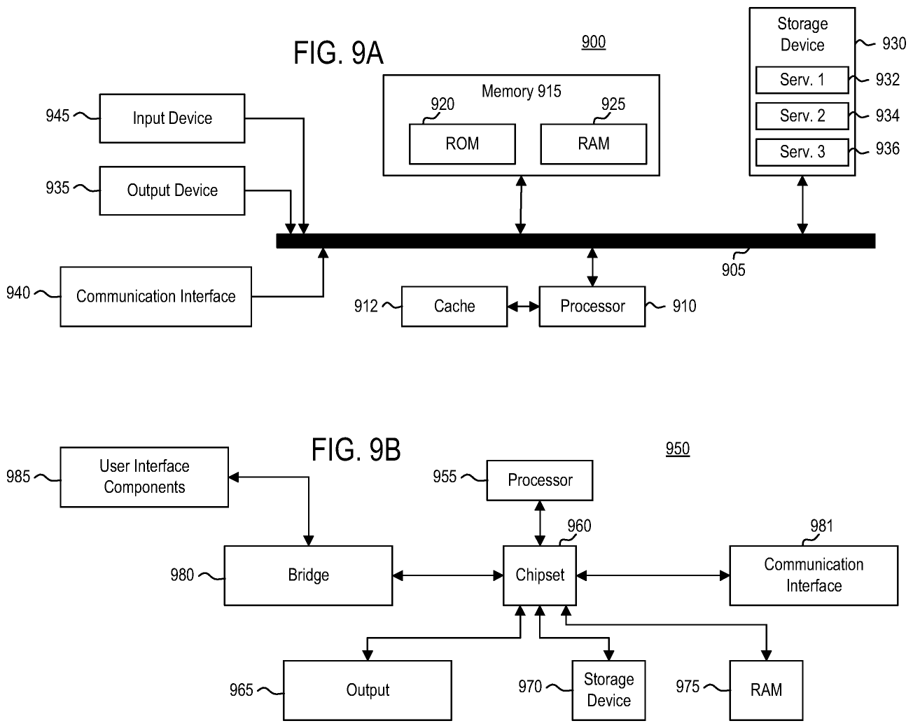

FIG. 9A illustrates a system bus computing system architecture (“system 900”), according to example embodiments. One or more components of system 900 may be in electrical communication with each other using a bus 905. System 900 may include a processor (e.g., one or more CPUs, GPUs or other types of processors) 910 and a system bus 905 that couples various system components including the system memory 915, such as read only memory (ROM) 920 and random access memory (RAM) 925, to processor 910. System 900 can include a cache of high-speed memory connected directly with, in close proximity to, or integrated as part of processor 910. System 900 can copy data from memory 915 and/or storage device 930 to cache 912 for quick access by processor 910. In this way, cache 912 may provide a performance boost that avoids processor 910 delays while waiting for data. These and other modules can control or be configured to control processor 910 to perform various actions. Other system memory 915 may be available for use as well. Memory 915 may include multiple different types of memory with different performance characteristics. Processor 910 may be representative of a single processor or multiple processors. Processor 910 can include one or more of a general purpose processor or a hardware module or software module, such as service 1 932, service 2 934, and service 3 936 stored in storage device 930, configured to control processor 910, as well as a special-purpose processor where software instructions are incorporated into the actual processor design. Processor 910 may essentially be a completely self-contained computing system, containing multiple cores or processors, a bus, memory controller, cache, etc. A multi-core processor may be symmetric or asymmetric.

To enable user interaction with the system 900, an input device 945 which can be any number of input mechanisms, such as a microphone for speech, a touch-sensitive screen for gesture or graphical input, keyboard, mouse, motion input, speech and so forth. An output device 935 can also be one or more of a number of output mechanisms known to those of skill in the art. In some instances, multimodal systems can enable a user to provide multiple types of input to communicate with system 900. Communications interface 940 can generally govern and manage the user input and system output. There is no restriction on operating on any particular hardware arrangement and therefore the basic features here may easily be substituted for improved hardware or firmware arrangements as they are developed.

Storage device 930 may be a non-volatile memory and can be a hard disk or other types of computer readable media that can store data that are accessible by a computer, such as magnetic cassettes, flash memory cards, solid state memory devices, digital versatile disks, cartridges, random access memories (RAMs) 925, read only memory (ROM) 920, and hybrids thereof.

Storage device 930 can include services 932, 934, and 936 for controlling the processor 910. Other hardware or software modules are contemplated. Storage device 930 can be connected to system bus 905. In one aspect, a hardware module that performs a particular function can include the software component stored in a computer-readable medium in connection with the necessary hardware components, such as processor 910, bus 905, output device 935 (e.g., a display), and so forth, to carry out the function.

FIG. 9B illustrates a computer system 950 having a chipset architecture that can be used in executing the described methods and generating and displaying a graphical user interface (GUI). Computer system 950 may be an example of computer hardware, software, and firmware that can be used to implement the disclosed technology. System 950 can include one or more processors 955, representative of any number of physically and/or logically distinct resources capable of executing software, firmware, and hardware configured to perform identified computations. One or more processors 955 can communicate with a chipset 960 that can control input to and output from one or more processors 955. In this example, chipset 960 outputs information to output 965, such as a display, and can read and write information to storage device 970, which can include magnetic media, and solid state media, for example. Chipset 960 can also read data from and write data to storage device 975 (e.g., RAM). A bridge 980 for interfacing with a variety of user interface components 985 can be provided for interfacing with chipset 960. Such user interface components 985 can include a keyboard, a microphone, touch detection and processing circuitry, a pointing device, such as a mouse, and so on. In general, inputs to system 950 can come from any of a variety of sources, machine generated and/or human generated.

Chipset 960 can also interface with one or more communication interfaces 981 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by one or more processors 955 analyzing data stored in storage device 970 or storage device 975. Further, the machine can receive inputs from a user through user interface components 985 and execute appropriate functions, such as browsing functions by interpreting these inputs using one or more processors 955.

It can be appreciated that example systems 900 and 950 can have more than one processor 910 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer- readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A method, comprising:
generating, by a computing system, a plurality of sets of training data, the plurality of sets of training data comprising portions of journal entries and inputs to mental health questionnaires corresponding to a plurality of patients;
generating, by the computing system, a prediction model to generate a health score of a patient, the health score indicative of a current mental health of the patient, by:
injecting tags into the portions of the journal entries that signal semantic tone and sentiment of each portion of each journal entry; and
learning, based on modified portions of the journal entries and the inputs to the mental health questionnaires, a relationship between the journal entries, the inputs, and a mental health of the patient;
receiving, by the computing system, input from a target patient, the input comprising target responses to the mental health questionnaires and target journal entries;
analyzing, by the computing system, the target journal entries using natural language processing to tag portions of each target journal entry with semantic tone and sentiment indicators; and
generating, by the computing system via the prediction model, a target health score for the target patient based on the target responses and the target journal entries.

2. The method of claim 1, wherein generating, by the computing system, the prediction model to generate the health score of the patient, further comprises:
encoding the portions of each journal entry with annotations from clinicians.

3. The method of claim 1, wherein generating, by the computing system, the prediction model to generate the health score of the patient, further comprises:
providing a clinician device with access to training results of the learning; and
receiving, from the clinician device, an adjustment to at least one of a weight or definition used by the prediction model.

4. The method of claim 1, wherein the prediction model is a neural network.

5. The method of claim 1, wherein receiving, by the computing system, the input from the target patient comprises:
prompting a patient device of the target patient to submit a target input to a mental health questionnaire; and
based on the prompting, receiving, from the patient device, the target input from the mental health questionnaire.

6. The method of claim 1, wherein receiving, by the computing system, the input from the target patient comprises:
prompting a patient device of the target patient to submit the target journal entry; and based on the prompting, receiving, from the patient device, the target journal entry.

7. The method of claim 6, wherein the target journal entry comprises one or more of a text based response, an audio based response, or an image based response.

8. A system, comprising:
a processor; and
a memory having programming instructions stored thereon, which, when executed by the processor, performs an operation comprising:
generating a plurality of sets of training data, the plurality of sets of training data comprising portions of journal entries and inputs to mental health questionnaires corresponding to a plurality of patients;
generating a prediction model to generate a health score of a patient, the health score indicative of a current mental health of the patient, by:
injecting tags into the portions of the journal entries that signal semantic tone and sentiment of each portion of each journal entry; and
learning, based on modified portions of the journal entries and the inputs to the mental health questionnaires, a relationship between the journal entries, the inputs, and a mental health of the patient;
receiving input from a target patient, the input comprising target responses to the mental health questionnaires and target journal entries;
analyzing the target journal entries using natural language processing to tag portions of each journal entry with semantic tone and sentiment indicators; and
generating, via the prediction model, a target health score for the target patient based on the target responses and the target journal entries.

9. The system of claim 8, wherein generating the prediction model to generate the health score of the patient, further comprises:
encoding the portions of each journal entry with annotations from clinicians.

10. The system of claim 8, wherein generating the prediction model to generate the health score of the patient, further comprises:
providing a clinician device with access to training results of the learning; and
receiving, from the clinician device, an adjustment to at least one of a weight or definition used by the prediction model.

11. The system of claim 8, wherein the prediction model is a neural network.

12. The system of claim 8, wherein receiving the input from the target patient comprises:
prompting a patient device of the target patient to submit a target input to a mental health questionnaire; and
based on the prompting, receiving, from the patient device, the target input from the mental health questionnaire.

13. The system of claim 8, wherein receiving the input from the target patient comprises: prompting a patient device of the target patient to submit the target journal entry; and based on the prompting, receiving, from the patient device, the target journal entry.

14. The system of claim 13, wherein the target journal entry comprises one or more of a text based response, an audio based response, or an image based response.

15. A non-transitory computer readable medium having instructions stored thereon, which, when executed by a processor, causes a computing system to perform operations, comprising:
generating, by the computing system, a plurality of sets of training data, the plurality of sets of training data comprising portions of journal entries and inputs to mental health questionnaires corresponding to a plurality of patients;
generating, by the computing system, a prediction model to generate a health score of a patient, the health score indicative of a current mental health of the patient, by:
injecting tags into the portions of the journal entries that signal semantic tone and sentiment of each portion of each journal entry; and
learning, based on modified portions of the journal entries and the inputs to the mental health questionnaires, a relationship between the journal entries, the inputs, and the mental health of the patient;
receiving, by the computing system, input from a target patient, the input comprising target responses to mental health questionnaires and target journal entries;
analyzing, by the computing system, the target journal entries using natural language processing to tag portions of the target journal entry with semantic tone and sentiment indicators; and
generating, by the computing system via the prediction model, a target health score for the target patient based on the target responses and the target journal entries.

16. The non-transitory computer readable medium of claim 15, wherein generating, by the computing system, the prediction model to generate the health score of the patient, further comprises:
encoding the portions of each journal entry with annotations from clinicians.

17. The non-transitory computer readable medium of claim 15, wherein generating, by the computing system, the prediction model to generate the health score of the patient, further comprises:
providing a clinician device with access to training results of the learning; and
receiving, from the clinician device, an adjustment to at least one of a weight or definition used by the prediction model.

18. The non-transitory computer readable medium of claim 15, wherein the prediction model is a neural network.

19. The non-transitory computer readable medium of claim 15, wherein receiving, by the computing system, the input from the target patient comprises:
prompting a patient device of the target patient to submit a target input to a mental health questionnaire; and
based on the prompting, receiving, from the patient device, the target input from the mental health questionnaire.

20. The non-transitory computer readable medium of claim 15, wherein receiving, by the computing system, the input from the target patient comprises:
prompting a patient device of the target patient to submit the target journal entry; and based on the prompting, receiving, from the patient device, the target journal entry.
